(19) United States
(12) Patent Application Publication
(10) Pub. No.: US 2007/0221869 A1
(43) Pub. Date: Sep. 27, 2007

(54) RADIOTHERAPY APPARATUS

(76) Inventor: Shi Peng Song, Shanghai (CN)

Correspondence Address: MATTHIAS SCHOLL, 14781 MEMORIAL DRIVE, SUITE 1319, HOUSTON, TX, 77079, US

(21) Appl. No.: 11/277,167

(22) Filed: Mar. 22, 2006

(51) Int. Cl. A61N 5/01; A61N 5/10

(52) U.S. Cl. 250/492.1; 378/65

(57) ABSTRACT

The invention described herein solves the problem of keeping the target-to-skin dose ratio high while simplifying the complex structure of the source and the radiation beams; thus, lowering the cost of the radiotherapy device. Taught are radiotherapy devices for treating a patient having a treatment volume comprising a drum with a first axis of rotation and a housing for a radiation source with a second axis of rotation, the first axis of rotation and a second axis of rotation intersecting at various angles at a focus point (O).

### BACKGROUND OF THE INVENTION

1. Field of the Invention

This invention relates to a radiotherapy apparatus for providing radiation therapy treatment to a patient, and specifically a radiotherapy apparatus having a rotatable radiation source.

2. Description of Related Art

Radiation therapy is widely used in treating certain types of cancers; it can be effective if a sufficient radiation dose is delivered to the tumor. However, if the effective radiation dose is delivered not only to the tumor but also to the surrounding healthy tissue, serious complications may result. Accordingly, devices and techniques have been developed to keep the target-to-skin dose ratio high. However, these devices and techniques generally rely on a complex structure of the source and the radiation beams and/or on multiple sources, and thus, have a high cost.

### BRIEF SUMMARY OF THE INVENTION

In one embodiment provided is a radiotherapy apparatus for treating a patient having a treatment volume comprising: a support frame; a drum having (i) an outer wall, (ii) an inner wall, (iii) a first axis of rotation, and (iv) a drum drive unit; a housing comprising a first treatment head and a radiation source, the housing having a second axis of rotation; and a means for rotating the housing with respect to the second axis of rotation; wherein the support frame is connected to the drum; the housing is rotatably connected to the support frame; the means for rotating said housing is connected to the housing; and the first axis of rotation and the second axis of rotation intersect at a focus point O.

In a class of this embodiment, the radiotherapy apparatus further comprises a collimator.

In a class of this embodiment, the radiotherapy apparatus further comprises a scanning means. In a subclass of this class, the scanning means is a CT apparatus, an X-ray apparatus, or a SPECT apparatus.

In a class of this embodiment, the radiotherapy apparatus further comprises a second treatment head heaving a high frequency and/or microwave radiation source.

In a class of this embodiment, the radiotherapy apparatus further comprises a bed; the scanning means detects the position of the treatment volume in real time. In a subclass of this class, the scanning means adjusts the bed so as to keep the treatment volume at the focus point (O). In a subclass of this embodiment, the scanning means comprises a scanning radiation source and a detector corresponding to said scanning radiation source.

In a class of this embodiment, the radiotherapy apparatus further comprises a bearing having an outer race and an inner race, the inner race being mechanically connected to the support frame and the outer race being mechanically connected to the housing.

In a class of this embodiment, the means for rotating the housing with respect to the second axis of rotation utilizes a motor and two or more gears to transfer rotation from the motor onto the housing.

In a class of this embodiment, the first axis of rotation and the second axis of rotation intersect substantially at a 90 degree angle.

In a class of this embodiment, the radiation source is disposed within the housing at a distance away from the second axis of rotation.

In a class of this embodiment, when the housing rotates around the second axis of rotation, the radiation source rotates around the second axis of rotation on a circular pathway.

In a class of this embodiment, the radiation source emits one or more radiation beams which intersect the second axis of rotation. In a subclass of this embodiment, one or more radiation beams intersect the second axis of rotation at an angle of from about 10 to about 50 degrees.

In a class of this embodiment, when the housing rotates about the second axis of rotation, the radiation beams form an area of conical shape centered about the second axis of rotation.

### BRIEF DESCRIPTION OF THE DRAWINGS

Below, the invention is explained in more detail with reference to drawings, in which:

FIG. 1 is a schematic representation of the radiotherapy apparatus in a front elevational view.

FIG. 2 is a schematic representation of the radiotherapy apparatus in a side elevational view.

FIG. 3 is a fragmentary top view of the housing and of the drum.

### DETAILED DESCRIPTION OF THE INVENTION

The invention described herein solves the problem of keeping the target-to-skin dose ratio high while simplifying the complex structure of the source and the radiation beams; thus, lowering the cost of the radiotherapy device.

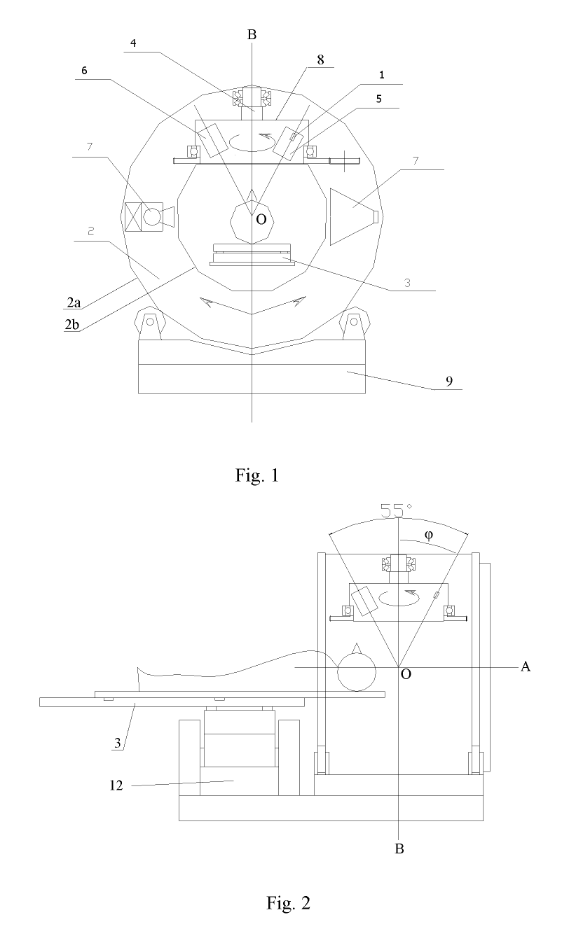

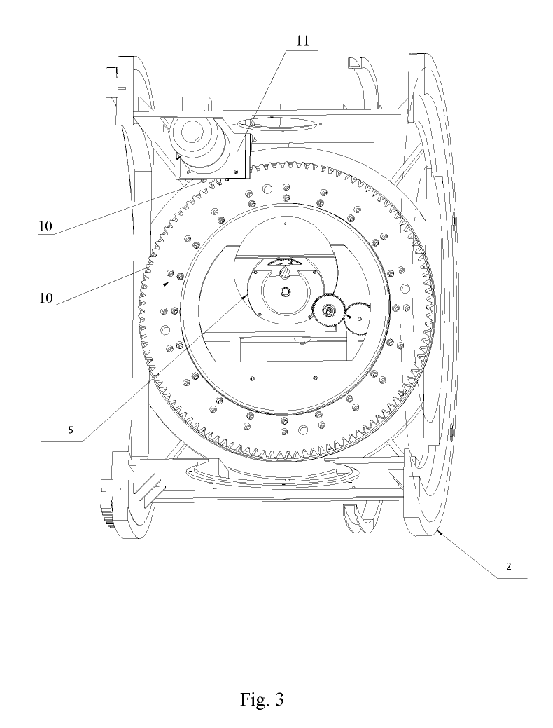

As illustrated in FIGS. 1-3, the invention provides a radiotherapy apparatus which has a therapeutically acceptable target-to-skin dose ratio comprising a support frame (4); a drum (2) having (i) an outer wall (2a), (ii) an inner wall (2b), (iii) a first axis of rotation (A), and (iv) a drum drive unit (9); a housing (8) comprising a first treatment head (5) and a radiation source (1), said housing (8) having a second axis of rotation (B); and means (11) for rotating said housing with respect to said second axis of rotation.

The drum (2) is mechanically connected to the drum drive unit (9). The drum drive unit (9) transfers rotation from a motor onto the drum (2). The drum (2) rotates around its first axis of rotation (A). The axis of rotation (A) is substantially horizontal, or it may be elevated from horizontal. Particularly, the rotary motion around the first axis of rotation may be transferred from a motor (e.g., an electric motor) onto said drum (2) via e.g., a gear train (e.g., a spur gear train, a parallel helical gear train, a double helical gear train, a bevel gear train, a worm gear train, etc.). One gear may be connected to or be a part of a motor, and the second gear may be connected to or be a part of the drum (2). Alternatively, rotary motion around the first axis of rotation may be transferred from a motor (e.g., an electric motor) onto said drum (2) directly, e.g., via rollers, wheels, balls, etc.

The drum (2) is mechanically connected to the support frame (4). The support frame (4) is, for example, an axle having two ends, wherein one end of the axle is connected to the outer wall of the drum (2a) and the other end of the axle is connected to the inner wall of the drum (2b). The connections of the axle to the drum may be via a clamp or a bracket. The connections of the axle to the drum may be also by welding, screwing, bolting, gluing, or any other means to inflexibly connect two mechanical parts.

The housing (8) is rotatably connected to the support frame (4), for example, via a bearing, or any other means to rotatably connect two mechanical parts. To connect the housing rotatably to the support frame by using a bearing, an outer race of the bearing is connected to the housing and an inner race of the bearing is connected to the support frame; or vice versa.

The means (11) for rotating the housing (8) with respect to the second axis of rotation (B) utilizes a motor and one or more gears (10) to transfer rotation from the motor onto the housing. Particularly, the rotary motion around the second axis of rotation (B) may be transferred from a motor (e.g., an electric motor) onto said housing (8) via e.g., a gear train (e.g., a spur gear train, a parallel helical gear train, a double helical gear train, a bevel gear train, a worm gear train, etc.). One gear may be connected to or be a part of a motor, and the second gear may be connected to or be a part of the housing (8). Alternatively, the first treatment head (5) may be directly rotatably connected to the means (11) for rotating said housing (8).

The first axis of rotation (A) and the second axis of rotation (B) intersect at a focus point (O). The first axis of rotation (A) and the second axis of rotation (B) may be normal to each other and intersect substantially at 90 degree angle.

The radiation source (1) is disposed within the housing (8) at a certain distance away from said second axis of rotation (B). In other words, when the housing rotates around the second axis of rotation (B), the radiation source (1) rotates around the second axis of rotation (B) on a circular pathway.

The radiation source (1) emits one or more radiation beams which intersect the second axis of rotation. The smaller of the angles (φ) formed by the intersection of a radiation beam with the second axis of rotation is from about 10 to about 50 degrees and more particularly from 20 to 30 degrees.

When the housing (8) rotates around the second axis of rotation, the radiation beams form an area of conical shape centered on the second axis of rotation (B).

The radiotherapy apparatus comprises further a treatment bed (3) on which a patient rests while undergoing radiation therapy. The bed is mechanically connected to a bed control system. The bed control system orients the bed in various positions with respect to the drum. The control system can move the bed linearly in and out of the drum. It can also raise and lower the front of the bed, the end of the bed, and each of the two sides of the bed, either simultaneously or separately.

The drum (2) may comprise further a scanning means (7) for inspecting the position of the patient and/or bed and/or treatment volume in real time. The scanning means may be, e.g., a CT apparatus, X-ray apparatus, or SPECT apparatus, or any other type of apparatus suitable for this purpose.

The scanning means (7) detects the position of the patient and/or treatment volume in real time, displays the position on a display (e.g., computer monitor), and adjusts the bed so as to keep the treatment volume in focus and to keep the peak-to-skin dose ratio at a relatively constant level. Real-time monitoring of radiation treatment can be achieved; for example, the bed may counteract the movement of the treatment volume during patient's breathing so as to keep it in place with respect to the focus point (O).

The means for rotating the housing (11), and/or the bed control system (12), and/or the drum drive unit (9) may be connected to the scanning means (7).

The housing (8) may comprise additionally a second treatment head (6) heaving a high frequency and/or microwave radiation source. The high frequency radiation beams emitted by the high frequency radiation source and/or microwaves emitted by the microwave radiation source focus on the focus point (O). The high frequency source and/or microwave radiation source are utilized simultaneously with the radiation source; this increases the temperature of the tumor cells and improves the efficiency of eradicating tumor cells.

The drum (2) rotates around the first axis of rotation (A) while the housing rotates around the second axis of rotation. This increases the area of the skin exposed to radiation while keeping the overall target-to-skin dose constant.

The radiotherapy apparatus may additionally comprise a collimator. The collimator is used to focus the radiation beams emitted from the radiation source onto focus point (O).

Treatment Procedure

1. The patient having a treatment volume undergoes a CT or X-ray scanning.

2. The scanned image is transferred to a Treatment Planning System (TPS) workstation for a doctor to set up a treatment schedule.

3. The treatment schedule prepared by the doctor is transferred to the radiotherapy apparatus of the present invention.

4. At the time of treatment, the scanning device detects the position of a tumor in real time and transfers the position information into a control system. The control system adjusts the position of the healing bed to keep the tumor in focus. The real-time monitoring and adjusting of the radiation treatment is achieved.

Definitions

The terms “peak-to-skin dose ratio” and “target-to-skin dose ratio,” as used herein, refer to the ratio between dose at the actual focus (target) and dose anywhere else in the (healthy) tissue—particularly at the skin interface. “Target-to-skin dose ratio,” is defined to be therapeutically acceptable, if it is commensurate with a reasonable benefit/risk ratio.

The terms “radioactive source,” “radiation source,” and “source,” as used herein, refer to sources of electromagnetic radiation useful for treating cancers and/or tumors in a patient in need of such treatment. A conventional radiation therapy system utilizes X-radiation energies in excess of 1 MeV. State-of-the-art therapy systems generate MegaVolt X-Radiation using linear accelerators. However, contemplated radiation sources for use in the devices and methods according to the invention described herein are not limited to X-ray sources, incl. linear accelerators, and may include without limitation other sources, such as, e.g., neutron sources, gamma ray sources, nuclear particle sources, ion sources, electron sources, proton sources, microwave sources, and radio frequency sources. Radiation sources, as used herein, also include radioactive isotopes.

The term “scanning radioactive source,” and “imaging radioactive source” as used herein, refer to a source of electromagnetic radiation useful for providing an image of the patient and/or the treatment volume. Scanning radioactive source is employed in a radiotherapy device in addition to a radioactive source. While the radioactive source produces electromagnetic radiation useful for treating cancer, a scanning radioactive source taken together with a scanning detector form a scanning device (scanning and imaging device) and provide a means for positioning the patient and the treatment volume for precise bombardment by radiation produced by a radiation source. The scanning radioactive source may be, e.g., an X-ray source. The scanning radioactive source may be the same source as the radiation source with provides radiation for treatment. The scanning device may employ CT as a method for producing images of the patient and the treatment volume.

The term “scanning means,” as used herein refers to a device that uses electromagnetic radiation to provide an image of the patient and/or the treatment volume. A scanning means generally employs a scanning radiation source and a scanning detector corresponding to the scanning radiation source. The radiation used to provide an image may be the same as the therapeutic radiation to treat cancer (e.g., X-rays-X-rays), or may be different (e.g., SPECT-X-rays). However, the amount of radiation needed to provide an image is generally much lower than the amount of radiation needed to affect treatment.

The term “CT” refers to X-ray Computed Tomography. It is a diagnostic imaging procedure that uses a combination of x-rays and computer technology to produce cross-sectional images (often called slices), both horizontally and vertically, of the body. A CT scan shows detailed images of any part of the body, including the bones, muscles, fat, and organs. CT scans are more detailed than standard X-rays. In computed tomography, the x-ray beam moves in a circle around the body. This allows many different views of the same organ or structure. The x-ray information is sent to a computer that interprets the x-ray data and displays it in a two-dimensional (2D) form on a monitor.

The term “SPECT” refers to Single Photon Emission Computed Tomography. It is a nuclear medicine tomographic imaging technique using gamma rays. It is very similar to conventional nuclear medicine planar imaging using a gamma camera. However, it is able to provide true 3D information. This information is typically presented as cross-sectional slices through the patient, but can be freely reformatted or manipulated as required.

The term “treatment volume,” as used herein, refers to that portion of the body of a patient in the need of radiotherapy, e.g., a cancer, a lesion, and/or a tumor.

The term “real-time” as referring to a scanning device means that the image of the patient and the treatment volume is produced at the time the radiation treatment is administered. Thus, any movements or changes in the position of the patient and/or the treatment volume are immediately registered, and the direction and/or shape of the radiation beam used for radiation treatment is altered immediately, if appropriate, so as to maintain the target-to-skin dose ratio.

The term “housing,” as used herein, refers to a support to which are attached a first treatment head (5) having a radiation source (1), and optionally a second treatment head (6).

The term “means for rotating the housing,” as used herein, refers to a means (11) for rotating the housing (8) with respect to the second axis of rotation (B). Various means to transfer rotation are known in the art. For example, rotation can be transferred onto the housing (e.g., from a motor) via a gear train. One gear may be connected to or be a part of a motor, and the second gear may be connected to or be a part of the housing (8). The gears may be spur gears, parallel helical gears, double helical gears, bevel gears, worm gears, or any other type of gears.

The terms “treatment bed” and “bed,” as used herein, refer to a support on which a patient rests while undergoing radiation therapy.

The term “collimator,” as used herein refers to an equipment in which it is desired to permit only beams of radiation emanating along a particular path to pass beyond a selected point or plane. Collimators are frequently used in radiation imagers to ensure that only radiation beams emanating along a direct path from the known radiation source strike the target (treatment volume, detector, etc). Collimators are described for example in US Application Publication No. US20060054841.

Whereas, particular embodiments of the invention have been described above for purposes of illustration, it will be evident to those skilled in the art that numerous variations of the details may be made without departing from the invention as defined in the appended claims.

What is claimed is:

1. A radiotherapy apparatus for treating a patient having a treatment volume comprising:
a support frame;
a drum having (i) an outer wall, (ii) an inner wall, (iii) a first axis of rotation, and (iv) a drum drive unit;
a housing comprising a first treatment head and a radiation source, said housing having a second axis of rotation; and
means for rotating said housing with respect to said second axis of rotation; wherein
said support frame is connected to said drum;
said housing is rotatably connected to said support frame;
said means for rotating said housing is connected to said housing; and
said first axis of rotation and said second axis of rotation intersect at a focus point 0.

2. The radiotherapy apparatus of claim 1 further comprising a collimator.

3. The radiotherapy apparatus of claim 1 further comprising a scanning means.

4. The radiotherapy apparatus of claim 3 wherein said scanning means is a CT apparatus, an X-ray apparatus, or a SPECT apparatus.

5. The radiation apparatus of claim 1 further comprising a second treatment head heaving a high frequency source and/or a microwave radiation source.

6. The radiation apparatus of claim 3 further comprising a bed, wherein said scanning means detects the position of the treatment volume in real time, and optionally adjusts said bed so as to keep the treatment volume at said focus point (O).

7. The radiation apparatus of claim 4 further comprising a bed, wherein said scanning means detects the position of the treatment volume in real time, and optionally adjusts said bed so as to keep the treatment volume at the focus point (O).

8. The radiation apparatus of claim 6 wherein said scanning means comprises a scanning radiation source and a detector corresponding to said scanning radiation source.

9. The radiation apparatus of claim 7 wherein said scanning means comprises a scanning radiation source and a detector corresponding to said scanning radiation source.

10. The radiation apparatus of claim 1 further comprising a bearing having an outer race and an inner race, said inner race being mechanically connected to said support frame and said outer race being mechanically connected to said housing.

11. The radiation apparatus of claim 1 wherein said means for rotating said housing with respect to the second axis of rotation utilizes a motor and two or more gears to transfer rotation from the motor onto said housing.

12. The radiation apparatus of claim 1 wherein said first axis of rotation and said second axis of rotation intersect at substantially a 90 degree angle.

13. The radiation apparatus of claim 1 wherein said radiation source is disposed within said housing at a distance away from said second axis of rotation.

14. The radiation apparatus of claim 1 wherein when said housing rotates around said second axis of rotation, said radiation source rotates around said second axis of rotation on a circular pathway.

15. The radiation apparatus of claim 1 wherein the radiation source emits one or more radiation beams which intersect the second axis of rotation.

16. The radiation apparatus of claim 15 wherein one or more radiation beams intersect said second axis of rotation at an angle of from about 10 to about 50 degrees.

17. The radiation apparatus of claim 15 wherein when said housing rotates about said second axis of rotation, said radiation beams form an area of conical shape centered about said second axis of rotation.
